(19) United States
(12) Patent Application Publication
(10) Pub. No.: US 2026/0345002 A1
(43) Pub. Date: Nov. 5, 2026

(54) COLOR SHIFT SUBJECTIVE REFRACTION SYSTEMS AND METHODS

(71) Applicant: 6 OVER 6 VISION LTD., Tel Aviv (IL)

(72) Inventors: Ofer Limon, Tel Aviv (IL); Alex Zlotnik, Tel Aviv (IL)

(21) Appl. No.: 19/656,146

(22) Filed: Apr. 23, 2026

Related U.S. Application Data
(60) Provisional application No. 63/798,174, filed on May 1, 2025.

(51) Int. Cl. A61B 3/14; A61B 3/103; G06T 3/40; G06T 5/92

(52) U.S. Cl. CPC A61B 3/14; A61B 3/103; G06T 3/40; G06T 5/92; G06T 2207/10024

(57) ABSTRACT

Methods and systems, including computer-implemented methods and systems, are described that test visual acuity and that can be used to determine parameters of an eye of a test subject, such as by performing a refraction of the eye. A target image is displayed to the test subject including a first spectral color, subjective recognition of visual elements of the target image is detected, a second target image is displayed to the subject including a second spectral color, subjective recognition of visual elements of the second target image is assessed, and at least one eye parameter is determined based on a distance and color properties of the second target image.

### CROSS-REFERENCE TO RELATED APPLICATION(S)

This application claims priority to U.S. Provisional Application No. 63/798,174, filed 1 May 2025, the entire disclosure of which is hereby incorporated by reference.

### TECHNICAL FIELD

The present disclosure generally relates to systems and methods for conducting refraction procedure in an optometric examination and particularly to systems and methods for conducting examinations for measuring refractive error.

### BACKGROUND

Refraction procedures in optometric examinations are used to identify various conditions such as refractive error of the eye. The refractive error of an eye (ametropia) is a defect in the ability of the eye to focus parallel rays of light from distant objects on the retina. A distant object is typically viewed at six meters or more from the tested eye. The object used is typically a character such as a letter, number, etc. of a predefined size presented over a board or a screen. Refractive error types include myopia, hyperopia, and astigmatism.

The refractive error of an eye may be evaluated using subjective or objective methods. Objective methods require a professional examiner to conduct the examination of the test subject's eye, using special equipment designed to determine the test subject's refractive status and prescribe the appropriate lens (glasses lens and/or contact lens) for each eye. Objective refraction may not require any test subject responses. The test subject has a passive role while his/her refractive error is evaluated by various possible objective methods and means. Auto refraction and retinoscopy are the most common objective methods. Methods such as photo refraction and aberrometry are also available and used. The less accurate objective refraction usually precedes the subjective result that is used to define the final prescription.

Subjective methods usually require the test subject to answer specific questions regarding the clarity of letters/targets presented on a chart at a distance of several meters (e.g., 6 meters). The examiner interposes lenses of various dioptric powers at a distance of approximately 12 mm from the test subject's eye, and the test subject is required to discern changes in the clarity of the characters by answering forced choice questions. Typically, the test subject is asked which of two lens presentations provides the better acuity.

Subjective refraction seeks to find the spherical power and the cylindrical power and axis. Most optometrists and ophthalmologists will use a cross cylinder in conjunction with a trial frame or phoropter in order to determine the cylindrical power and axis. The subjective refraction requires the examiner to interpret the test subject's responses.

### SUMMARY

One aspect of the present disclosure relates to a computer-implemented method for interactively determining an ocular parameter of an eye of a test subject, wherein the method comprises: displaying a first target image to a test subject, with the first target image including a first visual element comprising a first spectral color; determining that the test subject recognizes a property of the first visual element of the first target image at a distance from the display; displaying a second target image to the test subject, the second target image including a second visual element comprising a second spectral color; determining that the test subject does not recognize a property of the second visual element of the second target image at the distance from the display; and determining a parameter of an eye of the test subject based on the distance and the second spectral color.

The first visual element may comprise a set of lines having a first brightness and a first background having a second brightness, the first brightness being different from the second brightness. In some examples, the first brightness is about ten times brighter than the second brightness. In some examples, the second visual element comprises a second set of lines having a third brightness and a second background having a fourth brightness, the third brightness being different from the fourth brightness. In some examples, the second visual element comprises a border portion surrounding the second background, the border portion having a border brightness different from the fourth brightness.

In some examples, the first visual element comprises at least two separate elements set on a background and a border surrounding the background, the background having a first brightness, the border having a second brightness different from the first brightness. In some examples, the border has a different spectral color relative to the at least two separate elements.

In some examples, the first spectral color of the first visual element is substantially gray. The second spectral color of the second visual element may be a blue-shifted wavelength relative to the first spectral color of the first visual element. In some examples, the second spectral color shifts dioptric power of the second target image relative to the first target image.

In some examples, the property of the first visual element is a separation between two parts of the first visual element, and wherein the property of the second visual element is a separation between two parts of the second visual element.

In some cases, the method may further comprise, prior to displaying a second target image to the test subject, displaying an intermediate target image to the test subject, with the intermediate target image including a third visual element comprising a third spectral color; and determining that the test subject recognizes a property of the third visual element of the intermediate target image at the distance from the display.

In some examples, the method may further comprise determining the distance from the display, scaling a size of the first target image according to the distance, and scaling a contrast between portions of the first visual element according to the distance.

In some examples, the second spectral color comprises a blue: red ratio in a range of about 4:1 to about 20:1 in RGB values.

In some examples, the second spectral color comprises a blue: green ratio in a range of about 3:1 to about 5:1 in RGB values.

Another aspect of the disclosure relates to an apparatus for interactively measuring an ocular parameter, the apparatus comprising: a display configured to display to a test subject a user interface, a first target image, and a second target image; a sensor operable to detect a distance between the display and the test subject; a processor; a non-transitory, computer-readable memory device comprising instructions encoded thereon that, when executed by the processor, cause the processor to: trigger the display to show a first target image to a test subject, the first target image including a first visual element comprising a first spectral color; determine, via the sensor, a distance from the display to the test subject; determine, via the user interface, that the test subject recognizes a property of the first visual element of the first target image at the distance; trigger the display to show a second target image to the test subject at the distance, the second target image including a second visual element comprising a second spectral color; determine, via the user interface, that the test subject does not recognize a property of the second visual element of the second target image at the distance from the display; and determine a calculated parameter of an eye of the test subject based on the distance and the second spectral color.

In some examples, the first visual element comprises a set of lines having a first brightness and a first background having a second brightness, with the first brightness being different from the second brightness.

Yet another aspect of the disclosure relates to a non-transitory computer-readable storage medium comprising computer-executable instructions operable to, when executed by at least one computer processor, enable the at least one computer processor to cause at least one computing device to: display a first target image to a test subject, with the first target image including a first visual element comprising a first spectral color; determine a distance from the display to the test subject; determine that the test subject recognizes a property of the first visual element of the first target image at the distance; display a second target image to the test subject at the distance, with the second target image including a second visual element comprising a second spectral color; determine that the test subject does not recognize a property of the second visual element of the second target image at the distance from the display; and determine a calculated parameter of an eye of the test subject based on the distance and the second spectral color.

In some examples, the first visual element comprises a set of lines having a first brightness and a first background having a second brightness, the first brightness being different from the second brightness.

The above summary of the present invention is not intended to describe each embodiment or every implementation of the present invention. The Figures and the detailed description that follow more particularly exemplify one or more preferred embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings and figures illustrate a number of exemplary embodiments and are part of the specification. Together with the present description, these drawings demonstrate and explain various principles of this disclosure. A further understanding of the nature and advantages of the present invention may be realized by reference to the following drawings. In the appended figures, similar components or features may have the same reference label.

FIG. 1 is a block diagram of a computing system.

FIG. 2 is a block diagram of a method for determining an eye parameter.

FIGS. 3A-3C are schematic diagrams of a testing system for determining eye parameters according to the method of FIG. 2.

FIGS. 4A-4B illustrate an example target image for a visual acuity test.

FIGS. 5A-5B illustrate an example target image for a visual acuity test.

FIGS. 6A-6B illustrate an example target image for a visual acuity test.

FIG. 7 is a block diagram of a method for determining an eye parameter.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

Not all test subjects can afford the time and expense of visiting an eyecare professional to receive refraction testing (subjective or objective). Methods have been devised to allow a test subject to complete at least preliminary refraction tests without requiring the presence of an eyecare professional, such as by displaying various images (e.g., shapes, lines, letters, etc.) to a test subject using common electronic devices such as smartphones, computer tablets, and personal computers. The target images may be moved relative to the test subject until the images are at a maximum distance of best acuity (MDBA), as subjectively determined by the test subject viewing the images. The distance between the test subject and the device at the MDBA can be used to determine characteristics of the eye of the test subject (e.g., the compound lens characteristics) including spherical power, cylindrical power, and cylindrical axis. In some examples, this distance can be determined at least in part via range-finding sensors of the electronic device. The eye characteristics can be used to design and create corrective lenses (e.g., eyeglasses or contact lenses) with the help of eyecare professionals.

Test subjects often face a challenge of being able to move the electronic device to a sufficient distance while a test image is being shown. For some test subjects, the MDBA may be of sufficient length that it is difficult to find a room having a size sufficient to contain the test subject and the electronic device while at the MDBA, especially when a test subject wishes to take the vision test while in a residence, office, or other relatively small indoor structure. Thus, the test subject may have to inconveniently find a large area to complete the test.

Embodiments of the present disclosure relate to systems and methods for improving the administration of a subjective refraction. Using embodiments disclosed herein, a test subject can complete a test by initially being presented a target image including a first visual element comprising a first spectral color (e.g., gray), moving the target image to a maximum distance (e.g., their maximum preferred distance or the maximum distance available to them in a room), and, if the MDBA is still not reached, viewing a modified target image showing a second visual element comprising a second spectral color (e.g., blue).

Due to the properties of chromatic aberration of the eye, the shifted target image becomes more difficult to discern at the maximum distance than would otherwise be the case, so the test subject's effective MDBA (e.g., his or her lens power required to discern the intended visual elements of the target image as if it were at the actual distance indicated by the effective MDBA) can be determined even without moving the target image from the actual, physical maximum distance. The dimensions of the room in which the test subject receives the test can be, in effect, “stretched” without the test subject having to move further away from the display. For example, if the dimensions of a room limit a test to −1.00 diopter (D), chromatic modification/color shifting of the target image can be used to extend the test to about −1.25 D and/or −1.50 D. Thus, embodiments of the present disclosure can extend testing to low myopic ranges (e.g., between about −0.50 and about −3.00 diopters) where tests without color shifting would not be able to accurately find subjective distance-based acuity values such as the MDBA.

The present description provides examples, and is not limiting of the scope, applicability, or configuration set forth in the claims. Thus, it will be understood that changes may be made in the function and arrangement of elements discussed without departing from the spirit and scope of the disclosure, and various embodiments may omit, substitute, or add other procedures or components as appropriate. For instance, the methods described may be performed in an order different from that described, and various steps may be added, omitted, or combined. Also, features described with respect to certain embodiments may be combined in other embodiments.

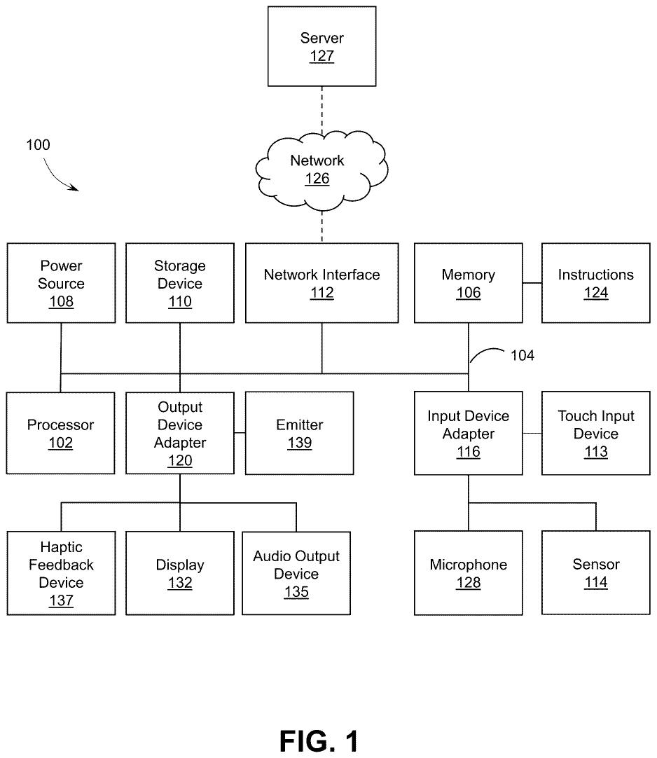

FIG. 1 shows a high-level block diagram of a computer system 100 that can be used to implement embodiments of the present disclosure. In various embodiments, the computer system 100 can comprise various sets and subsets of the components shown in FIG. 1. Thus, FIG. 1 shows a variety of components that can be included in various combinations and subsets based on the operations and functions performed by the system 100 in different embodiments. It is noted that, when described or recited herein, the use of articles such as “a” or “an” is not considered to be limiting to only one, but instead is intended to mean one or more unless otherwise specifically noted herein.

The computer system 100 can comprise a central processing unit (CPU) or processor 102 connected via a bus 104 for electrical communication to a memory device 106, a power source 108, an electronic storage device 110, a network interface 112, an input device adapter 116, and an output device adapter 120. For example, one or more of these components can be connected to each other via a substrate (e.g., a printed circuit board or other substrate) supporting the bus 104 and other electrical connectors providing electrical communication between the components. The bus 104 can comprise a communication mechanism for communicating information between parts of the system 100.

The processor 102 can be a microprocessor or similar device configured to receive and execute a set of instructions 124 stored by the memory 106. The memory 106 can be referred to as main memory, such as random access memory (RAM) or another dynamic electronic storage device for storing information and instructions to be executed by the processor 102. The memory 106 can also be used for storing temporary variables or other intermediate information during execution of instructions executed by the processor 102. The processor 102 can include one or more processors or controllers, such as, for example, a CPU for the computing device 100 in general and a touch controller or similar sensor or I/O interface used for controlling and receiving signals from the display 132 and any other sensors being used (e.g., 128, 114, 113). The power source 108 can comprise a power supply capable of providing power to the processor 102 and other components connected to the bus 104, such as a connection to an electrical utility grid or a battery system.

The storage device 110 can comprise read-only memory (ROM) or another type of static storage device coupled to the bus 104 for storing static or long-term (i.e., non-dynamic) information and instructions for the processor 102. For example, the storage device 110 can comprise a magnetic or optical disk (e.g., hard disk drive (HDD)), solid state memory (e.g., a solid state disk (SSD)), or a comparable device.

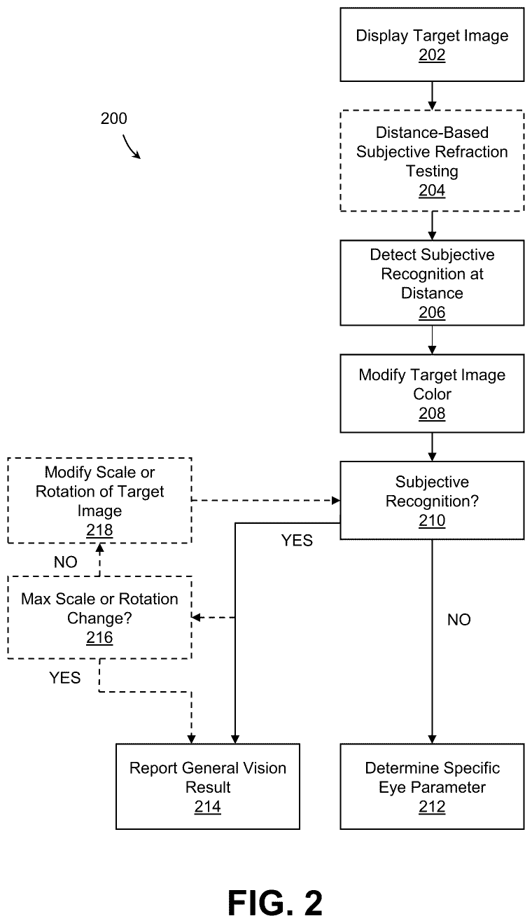

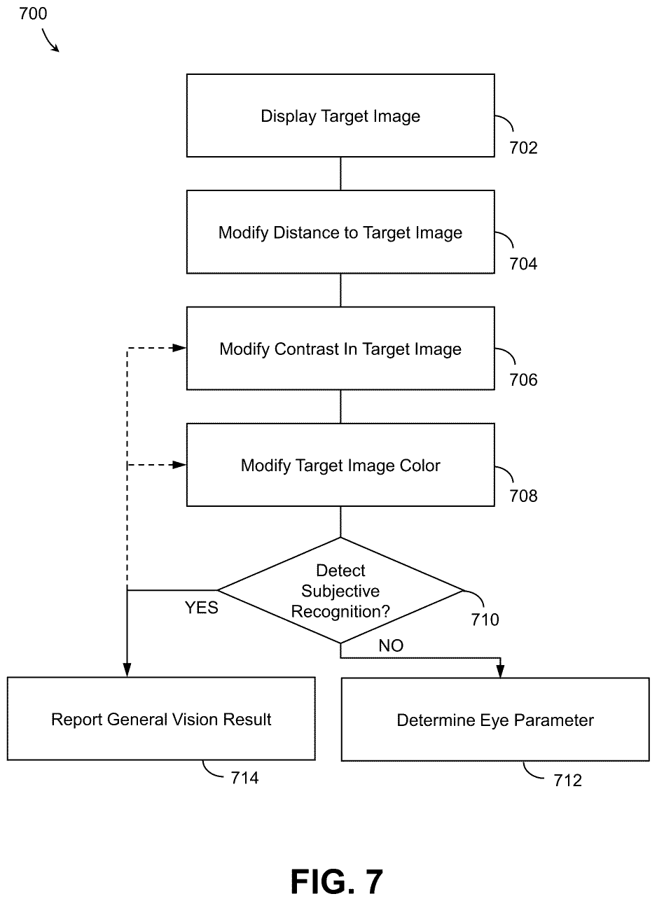

The instructions 124 can comprise information for executing processes and methods using components of the system 100. Such processes and methods can include, for example, the methods 200, 700 described in connection with other embodiments elsewhere herein, including, for example, the methods and processes described in connection with FIGS. 2 and 7.

The network interface 112 can comprise an adapter for connecting the system 100 to an external device via a wired or wireless connection. For example, the network interface 112 can provide a connection to a computer network 126 such as a cellular network, the Internet, a local area network (LAN), a separate device capable of wireless communication with the network interface 112, other external devices or network locations (e.g., a server 127), and combinations thereof. In one example embodiment, the network interface 112 is a wireless networking adapter configured to connect via WI-FI®, BLUETOOTH®, BLE, Bluetooth mesh, or a related wireless communications protocol to another device having interface capability using the same protocol. In some embodiments, a network device or set of network devices in the network 126 can be considered part of the system 100. In some cases, a network device can be considered connected to, but not a part of, the system 100. In some examples, a network-connected server 127 can include a separate processor and memory device configured to receive information from a computing device used by the test subject and to perform one or more actions of methods described herein.

The input device adapter 116 can be configured to provide the system 100 with connectivity to various input devices such as, for example, a touch input device 113 (e.g., a touchscreen, touchpad, or resistive or capacitive touch sensing array), a keyboard or other peripheral input device, a microphone 128, a sensor 114 (e.g., an infrared (IR) sensor), one or more other sensors, related devices, and combinations thereof. In an example embodiment, the input device adapter 116 is connected to the touch input device 113 and configured to detect taps, touches, or gestures by a test subject on a surface of the computing system 100. In some configurations, the input device adapter 116 can include a touch controller or similar interface controller. The sensor 114 can be used to detect physical phenomena in the vicinity of the computing system 100 (e.g., light, sound waves, electric fields, forces, vibrations, etc.) and convert those phenomena to electrical signals. In some examples, the sensor 114 may be a distance metering sensor configured to sense time-of-flight (ToF) information of the output of an output device (e.g., emitter 139). The microphone 128 can be used to receive audible input from a test subject, such as when the test subject is not within an arm's reach distance of the display 132 or other input devices.

The output device adapter 120 can be configured to provide the system 100 with the ability to output information to a user, such as by providing visual output using one or more displays 132 (e.g., liquid crystal displays (LCDs), organic light emitting diode (OLED) displays, similar electronic display screens, and combinations thereof configured to present images to a user via pixels capable of changing their visible chromatic appearance (e.g., RGB pixels)), by providing audible output using one or more speakers 135, or providing haptic feedback sensed by touch via one or more haptic feedback devices 137. Other output devices can also be used. The processor 102 can be configured to control the output device adapter 120 to provide information to a user via the output devices connected to the adapter 120, such as visible or audible instructions, a user interface, or haptic feedback. In some examples, the output device adapter 120 can be connected to an emitter 139 configured to output a signal (e.g., visible light, ultraviolet light, infrared light, sound waves, ultrasonic waves, similar outputs, and combinations thereof) usable to detect a distance between a test subject and the display 132 (e.g., when sensed by sensor 114).

FIG. 2 illustrates elements of a method 200 of interactively calculating an ocular parameter of a test subject according to an embodiment of the present disclosure. The method 200 may be a computer-implemented method, wherein an electronic device, such as, for example, a computer system (e.g., 100), can be used to complete various actions, comparisons, and calculations to determine at least one parameter of an eye of a test subject. The method 200 can include displaying a target image, as indicated in block 202, optionally completing a distance-based subjective refraction test, as indicated in block 204, detecting subjective recognition of the target image at a distance, as indicated in block 206, modifying a target image color, as indicated in block 208, detecting whether the subject has subjective recognition with the modified color target, as indicated in block 210, and either determining an eye parameter, as indicated in block 212, or reporting a general vision result, as in block 214. Portions of the method 200 may be optional and not performed in every embodiment of determining ocular parameters according to the present disclosure. Additionally, in some examples, if subjective recognition is achieved in block 210, the test can include changing the scale or rotation of the target image, as indicated in blocks 216 and 218, before testing for subjective recognition again (block 210) or reporting a general vision result (block 214), as explained in further detail below.

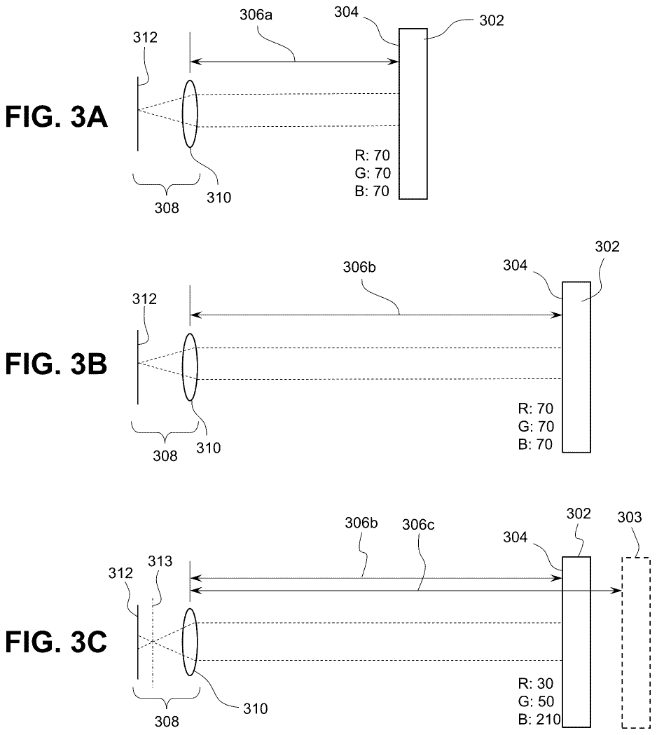

Displaying a target image, as indicated in block 202, can include triggering a display screen to show a target image to a test subject. For example, a display screen (e.g., a display 132 of system 100 of FIG. 1 or a display 304 of an electronic device 302 of FIG. 3A) can be controlled to show the target image via a surface of the device configured to be visible by the test subject. The target image can be displayed having visual characteristics (e.g., overall or average brightness, overall or average contrast, scaled dimensions, spectral colors/hues/filters, saturation, and vibrance) and visual elements or visual features (e.g., specific shapes, lines, borders, gradients, specific element brightnesses, and relative contrasts).

As used herein, red/green/blue (RGB) values of pixels on a display screen can lie in a scale/range of 0 to 255 for each component color, with 0 being minimum brightness (or off completely, depending on the type of display screen technology used) and with 255 being maximum brightness. Displays showing a target image can have a luminance/illuminated brightness (e.g., measured in nits) based on their backlight intensity (for backlit displays) or light output (for light emitting displays, such as OLEDs, micro-LEDs, and similar displays).

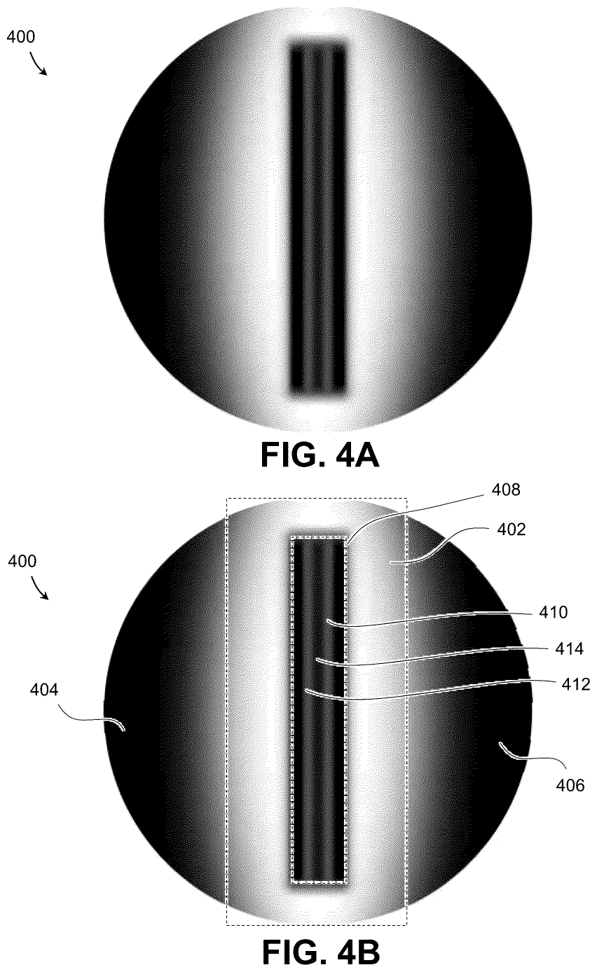

A first example target image 400 is illustrated in FIGS. 4A and 4B. The underlying target image 400 of FIG. 4A is the same as the target image 400 of FIG. 4B but is shown without annotations so that the image of FIG. 4A can accurately represent what a test subject would be displayed without occlusion of reference lines used for convenience in referring to portions of the image in the present disclosure.

The target image 400 may have a set of visual characteristics including an overall or average brightness, with a central region 402 of the image 400 having relatively high maximum brightness values (e.g., illuminated pixels having RGB values above about 230/230/230 (respectively) to produce white and gray in a range between about 140/140/140 and about 255/255/255) and a left region 404, a right region 406, and a center background box 408 having lower maximum brightness values (e.g., near about 0/0/0) due to containing black or nearly black pixels. In one example embodiment, the average RGB brightness of all pixels shown to a test subject via a display 304 can be about 70/70/70, as indicated in FIG. 3A. Central region 402 may be referred to as a border region or border portion of the image since it borders and surrounds center background box 408.

As used herein, the “intensity” of a target image refers to its average brightness, such as, for example, its average values of red, green, and blue across the target image in an RGB pixel-based display. Thus, in the example of FIG. 3A, the target image may have an intensity of 70 as that is the average of the three red, green, and blue component values across the entire image. Moreover, in the example of FIG. 3C, the target image on display 304 has an intensity of 96.67.

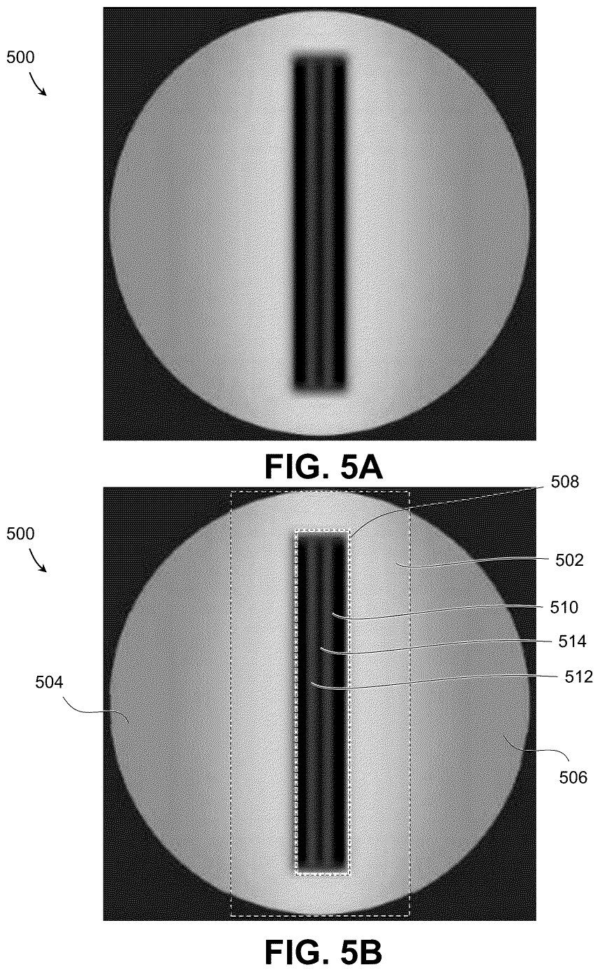

The target image 400 may include an overall or average contrast level defined by the relative brightness levels of the individual elements of the target image 400. The contrast can be defined as a ratio of different brightness values (or average brightness values). For instance, a contrast ratio of about 10:1 or more can be implemented between the parallel line features 410 and 412 (each of which has the same intensity and is separated from the other) and the darkest part of the center background box 408. A contrast ratio of about 6:1 can be implemented between each of the parallel line features 410, 412 and a parallel center field 414 lying between the parallel line features 410, 412. In some examples, the parallel line features 410, 412 may have an RGB blue-to-red ratio within a range of about 4:1 to about 20:1 in RGB values and may have an RGB blue-to-green ratio within a range of about 3:1 to about 5:1 in RGB values. See also FIGS. 5A-5B and their descriptions elsewhere herein. These ratios and values may provide sufficient chromatic shifting to ensure dioptric power of the test subject's eye is adequately affected by the color change while the testing procedure is administered by a system 100 and via method 200.

In some examples, the electronic device (e.g., system 100 or 302) may include an ambient light sensor, and ambient light brightness conditions can be measured and used to control contrast or intensity of a target image displayed. For instance, a bright ambient environment for the test subject can be detected, and the processor can cause the display to present a target image with greater brightness as compared to when a relatively dark ambient environment is detected. See also FIG. 7 and its related description below.

The target image 400 can be shown on a display having a defined scale or on-screen dimensions based on the type or characteristics of the display and the electronic device on which the target image 400 is shown. In some examples, the scale or on-screen dimensions can be partially based on an expected, estimated, or measured distance (e.g., 306a or 306b in FIGS. 3A-3C) between the test subject and the display. Thus, displaying a target image to a test subject (e.g., as in block 202) can include determining features and characteristics of a display and/or electronic device (e.g., 132, 100, 302) such as a model number, screen size, pixel density (e.g., pixels per millimeter or inch), aspect ratio, and similar properties so that the target image is presented with a known real-world size and appearance. The distance 306 between the test subject 308 and the display 304 can be determined via user input via a user interface, such as by the user inputting a distance value to the device 302 using an input device (e.g., buttons or touch screen), using visible gestures detected by an imaging device/camera, or using a microphone. In some examples, the distance can be determined using a distance metering device such as an infrared emitter and detector of the electronic device 302 (e.g., emitter 139 and sensor 114). In some examples, the distance can be estimated, such as by capturing at least one image of the test subject using a subject-facing imaging device (e.g., camera) of the electronic device 302 (or a camera connected to the input device adapter 116), detecting the size of features of the test subject (e.g., pupil diameters) or other reference objects (e.g., a credit card, ruler, or other common object having one or more known size dimensions), and determining the distance from the imaging device to the test subject by comparing the imaged size of the reference object or test subject features.

The distance between the test subject and the electronic device 302 can be used to scale the size and/or contrast of the target image 400 to meet desired parameters for visibility of portions of the target image 400. For example, a target image shown to the test subject with relatively small real-world dimensions (i.e., an image that is scaled down due to being relatively close to the user) can be presented with higher contrast, and a target image shown with relatively large real-world dimensions (e.g., an image that is scaled up due to being farther from the user) can be presented with lower contrast. See also FIG. 7 and its related descriptions below. When the target image is at a greater distance from the test subject, the test subject may thereby discern visual elements (e.g., 402, 410, 412, 414, 408) of the target image 400 with substantially the same visual acuity as a test subject at a closer distance to differently-scaled visual elements. In this way, the test subject's eyes' diopter, spherical power, cylindrical power, cylindrical axis, and other eye lens properties (e.g., properties of the eye's compound lens of crystalline and corneal lenses) can be determined even when the test subject is displayed images under different environmental circumstances.

As explained above, the target image 400 can be presented using a plurality of individual pixels or dots, each having spectral colors/hues/filters, saturation, and vibrance. Referring again to FIG. 2, displaying the target image (202) can comprise displaying a first target image or a first version or color setting of the target image. In some examples, a first target image presented to the test subject can comprise a substantially grayscale spectral color/hue/filter and saturation. Thus, the RGB values of the pixels, dots, or other displayed portions of the target image can be substantially equal to each other, such as the 70/70/70 RGB setting of FIG. 3A. As further explained below, the target image can have its spectral color features modified or the target image can be replaced by a different target image having different spectral color features as part of the method 200.

The target image 400 may also comprise a set of visual elements or visual features (e.g., specific shapes, lines, borders, and gradients). For example, the target image 400 may include a pair of lines (e.g., 410, 412), shapes (e.g., rectangular central background box 408), a line between the pair of lines (e.g., 414), a gradient between the central region 402 and side regions 404, 406, and an overall circular shape (i.e., the circular outline of the entire target image 400). These features may all be oriented relative to each other or relative to axes across the target image. Thus, visual elements may be parallel to each other (e.g., 410, 412, 414 are parallel to each other and parallel to the sides/edges of the central background box 408), surround each other (e.g., central region 402 surrounds center background box 408), etc. The entire target image 400 can be displayed at a desired angle, such as with the pair of lines 410, 412 being vertical (as in FIGS. 4A-4B) or rotated relative to the vertical direction at an angle (e.g., 45 degrees angularly offset from the position shown in FIGS. 4A-4B). Rotation of the target image 400 can allow testing cylindrical power of the test subject's eye for various angles, as further described below.

Referring again to FIG. 2, the method 200 may optionally include completing a distance-based subjective refraction test in block 204. An example distance-based subjective refraction test that can be used is described in U.S. patent application Ser. No. 17/361,095, which is hereby incorporated by reference in its entirety. To illustrate, a test subject may be presented the target image 400 and requested to provide input regarding whether they subjectively can visually resolve two parallel lines (e.g., 410, 412) at a first distance. The first distance can be a preset distance or a measured distance (e.g., measured in situations where the test subject is at a variable position relative to the target image). If the lines cannot be subjectively recognized, the test subject may move closer to the target image until the lines are recognized. On the other hand, if the lines can be subjectively recognized, the test subject may move relative to the target image farther away, testing at various incrementally increasing distances, until reaching a maximum distance, e.g., a maximum distance possible in a test environment due to physical constraints of the testing location or a maximum distance at which a certain display is able to present the target image at a desired size and position relative to the test subject. At that point, the distance-based subjective refraction testing (e.g., of block 204) can conclude. Modifying the distance may include providing instructions to the test subject to move the target image relative to the test subject, such as, for example, by having the test subject or an assistant move the device 302 to distance 306b from the test subject, which is greater than distance 306a. Thus, the test subject may be instructed to move the electronic device 302 from a first distance 306a between the test subject 308 and the display 304 to a second distance 306b between the test subject 308 and the display 304. The method 200 may include determining the distance 306a/306b (e.g., using a measurement or estimation method described above) or confirming the distance via test subject input. Thus, the method 200 can include positioning the target image at a predetermined distance from the test subject.

If, at maximum distance in the distance-based subjective refraction test, the test subject can still subjectively recognize the lines in the target image, the test subject is considered to subjectively recognize the target image at that distance, as indicated in block 206. Detecting subjective recognition, as indicated in block 206, can include requesting and receiving input from the test subject that the test subject recognizes a property of one or more visual elements of the target image when the target image is displayed at a particular distance. The recognition is referred to herein as “subjective recognition” since the user is expected to volunteer an answer to a prompt to indicate whether they personally believe they see or do not see the property of the one or more visual element of the target image. For target image 400, the test subject may be requested to indicate whether he or she recognizes the visual elements of two distinct lines 410, 412 that are separated by a center field 414 or whether he or she only sees one line (i.e., parts 410, 412, 414 blurred together). Other similar tests for binary recognition can be used, wherein the test subject is required to indicate whether they do or do not recognize or see a particular shape, configuration, separation, symbol, etc. Detecting subjective recognition can therefore include determining that the test subject sees the two lines 410, 412 distinct from each other.

Put another way, in some examples, when the test subject recognizes the tested property of the visual element of the target image (e.g., recognizes both lines 410, 412), the method 200 can include modifying the distance to the target image, as in block 204, until one of two conditions is met: first, the distance may be modified until the test subject does not recognize the property of the visual element anymore (e.g., due to being too far away for their eyes to recognize both lines) or second, the distance may be modified until the electronic device 302 is moved to a maximum distance from the test subject for the given testing environment and conditions (e.g., room size, lighting conditions, or nearby objects). The maximum distance may be a preset distance value (e.g., about two meters from the test subject) or may be based on input from the test subject (e.g., the test subject indicates the device can move no further from him or her in the test environment, such as if the test subject indicates that a wall is in the way or his or her arm cannot hold the device at any greater distance from his or her eye). In FIG. 3B, the maximum distance is indicated as distance 306b.

If the test subject provides input that he or she does not recognize the property of the visual element anymore before or upon reaching the maximum distance after completion of blocks 204/206, the electronic device 100/302 can determine the distance between the device and the test subject 308 and identify that distance as the maximum distance of best acuity (MDBA) of the test subject. The MDBA can be used to calculate the spherical power, cylindrical power, cylindrical axis, and other properties of test subject's eyes, as described, for example, in U.S. Pat. No. 10,898,071, issued 26 Jan. 2021, which is hereby incorporated by reference in its entirety.

If the test subject provides input that he or she recognizes the property of the visual element upon reaching the maximum distance in connection with blocks 204 and 206, e.g., the system 100 detects subjective recognition of the visual element(s) of the target image 400 at that distance, the method 200 can further proceed to display a second (e.g., a modified or different) target image to the test subject that includes a second (e.g., a modified or different) visual element comprising a second (e.g., a modified or different) spectral color. See block 208. In some examples, the target image can change from a first target image 400, shown in FIGS. 4A-4B, to a second target image 500, shown in FIGS. 5A-5B.

Like target image 400, the second target image 500 is shown without annotation in FIG. 5A, for clarity in description and to illustrate how the image would appear to a test subject and is shown with annotation in FIG. 5B. The second target image 500 may have different visual elements and/or visual characteristics of the displayed visual elements. In some examples, the second target image 500 can comprise identical (or substantially identical) shapes and dimensions as target image 400. For example, the second target image 500 can comprise the same positioning and size as the first target image 400 in the display, with the same size and dimensions for its central region 502, left region 504, right region 506, center background box 508, parallel line features 510, 512, and parallel center field 514.

The second target image 500 can include visual elements comprising a second spectral color. For instance, elements of the second target image 500 can be blue shifted in their RGB values (and accordingly in their visible wavelengths) to further test a change in diopter of the test subject. Chromatic aberration in the human eye occurs because the lens and cornea refract light differently depending on its wavelength. This means that light of different colors (wavelengths) does not focus at the same point on the retina. The refractive index of the eye's optical elements is higher for shorter wavelengths (blue/violet light) than for longer wavelengths (red light). As a result, blue light shifts focus of an image toward a position in front of the retina, and red light shifts focus of an image toward a position behind the retina. This difference in focus due to wavelength dependency leads to what is called longitudinal chromatic aberration.

The refractive power of the eye, measured in diopters (D), is the inverse of the focal length in meters. Since the focal length changes slightly for different wavelengths of light, the effective refractive power also changes. For example, when viewing blue light, the focal length decreases (higher refractive index), resulting in a slightly higher diopter value. When viewing red light, the focal length increases (lower refractive index), resulting in a slightly lower diopter value. The difference in diopter between blue and red light in a typical human eye is around 1.5 diopters. This change can affect sharpness for multicolored or monochromatic images if the eye cannot fully accommodate the differences. Changing between white and blue light, such as in embodiments described herein, can change diopter by about −0.5 D to about −0.75 D.

To illustrate how this can be used in connection with method 200, FIGS. 3A-3C show a test subject 308 having an ocular lens 310 and retina 312. If, at various distances 306a, 306b, the test subject can focus the light of target image 400 from the display 304 on the retina 312, as shown in FIGS. 3A-3B, the target image 400 can be blue-shifted (e.g., to have average brightness of RGB values of 30/50/210, as indicated in FIG. 3C) to cause the focal point 313 of the target image to lie in front of the retina 312. In some examples, the target image can be shifted from standard black and white or grayscale, with median densities in the greens of about 513 nanometers (nm) average wavelength toward blue light at about 450 nm to about 480 nm average wavelength. This shift in the focus of the image toward the front of the retina can be configured to give the second target image 500 a difficulty of resolving elements in the image as if being farther away from the test subject (e.g., as if the electronic device were positioned at the position of device 303, at effective distance 306c, instead of the electronic device 302 being positioned at its actual distance 306b from the test subject). The change in the spectral color displayed to the test subject can therefore simulate movement of the electronic device 302 when the test subject is testing their vision in an environment where actual movement of the device is not possible or convenient.

In the example of second target image 500, various visual elements of the image can be spectrally shifted differently relative to each other. For example, the parallel line features 410, 412 can be shifted from RGB values of about 80/80/80 to line features 510, 512 with RGB values of about 20/60/200. Thus, for those line features 410, 412, the average red value is 300% greater in the first target image 400 relative to the second target image 500, the average green value is 33% greater in the first target image 400, and the average blue value is 250% greater in the second target image 500. The brightness of the red and green portions of the color is decreased, and the brightness of the blue portion of the color is increased, for an overall different average brightness as compared to the line features 410, 412 of FIG. 4A. The parallel center field 414 can shift from RGB values of about 30/30/30 to center field 514 with RGB values of about 0/20/70, again changing the brightness of the center field 514 as compared to center field 414. Thus, for the center field 414, the average red value is about 3,000% greater (i.e., moving from a value of 30 in center field 414 to a value of less than one in center field 514) in the first target image 400, the average green value is 50% greater in the first target image 400, and the average blue value is 133% greater in the second target image 500. Accordingly, the spectral shift for the parallel line features 510, 512 is different from the spectral shift for the parallel center field 514.

In another example, the brightest part of the central region 402 can shift from RGB values of about 250/250/250 to brightest RGB values of central region 502 of about 120/250/250. Accordingly, only the red values may be shifted. Left and right regions 404, 406 can be shifted from average RGB values of about 20/20/20 or less to left and right regions 504, 506 with average RGB values of about 85/190/195, increases of 425%, 950%, and 975%, respectively. Thus, various parts of the first target image 400 can be spectrally shifted differently from other parts of the image. For this reason, the visual acuity of the test subject can be tested relative to individual parts of the target image in different ways, with more blue-shifted parts of the image being tested at a greater virtual or perceived distance/diopters/vergence and therefore being more difficult for the test subject to resolve. A test subject that is capable of resolving a separation between the two parallel line features 410, 412 at distance 306b may be incapable of resolving the separation of parallel line features 510, 512 at distance 306b.

The degree or magnitude of the spectral shift can affect the test subject's ability to resolve or recognize the visual elements of the second target image 500. Thus, as shown in block 210, the method 200 may include determining whether the test subject has subjectively recognized one or more elements of the target image 500 (after colors have been shifted). In some examples, if the system detects that the test subject can still subjectively recognize properties of the second visual element (e.g., the test subject can still resolve the parallel line features 510, 512 separately, as indicated by their input), the color of the second target image 500 can be further modified and adjusted (e.g., further blue-shifted to a different diopter), as indicated in block 208. The test subject can then be re-tested on whether he or she detects subjective recognition of the second visual element after this further adjustment (e.g., through repetition of block 210). This process can iterate as needed until the test subject no longer subjectively recognizes the property of the visual element of the target image being tested, i.e., there is no subjective recognition.

In some examples, if the system detects that the test subject can still subjectively recognize properties of the second visual element (e.g., the test subject can still resolve the parallel line features 510, 512 separately, as indicated by the test subject's input), the system can determine whether the target image is being displayed at its maximum scale, as indicated in block 216. If not at maximum scale, the scale can be modified (e.g., the scale of the target image 500 can be increased as displayed to the test subject), and the test subject's subjective recognition of the image is tested again. As long as the test subject subjectively recognizes the image, this process (through blocks 210, 216, and 218) can iteratively continue until maximum scale is reached, at which point a general vision result can be recorded or reported, as indicated in block 214.

Similarly, in some examples, if the system detects that the test subject can still subjectively recognize properties of the second visual element (e.g., the test subject can still resolve the parallel line features 510, 512 separately, as indicated by the test subject's input), the system can determine whether the target image is being displayed at its maximum rotation, as indicated in block 216. In this context, the rotation of the target image 500 refers to the angle of its rotated positioning relative to the display on which the test subject views the target image. For example, if the parallel lines 510, 512 of FIGS. 5A-5B are considered to have zero degrees of rotation in the target image 500 as shown in FIGS. 5A and 5B, the target image (or at least portions thereof) can be rotated in connection with block 218 by an incremental rotation angle between tests, e.g., as the rotation of the target image is modified each time block 218 is performed. Rotation of the target image can enable a test subject to improve their perception and the subjective clarity of the target image 500, especially for test subjects with different kinds of astigmatism. In some examples, the image can be rotated about 15 to about 20 degrees per iteration. In some examples, the increment of rotation can be about 35 degrees to about 45 degrees per iteration.

Eventually, a maximum rotation can be reached, such as 180 degrees of rotation, at which the target image 500 looks the same as originally presented to the test subject. In some examples, 90 degrees of rotation is considered the maximum rotation. Thus, if, during a test, the target image is not yet at maximum rotation in block 216, the rotation can be modified (e.g., the rotation of the target image 500 can be incremented as displayed to the test subject), and the test subject's subjective recognition of the image is tested again in block 210. As long as the test subject subjectively recognizes the image, this process (through blocks 210, 216, and 218) can iteratively continue until maximum rotation is reached, at which point a general vision result can be recorded or reported, as indicated in block 214.

The general vision result of block 214 can include recording, sending, announcing, reporting, or providing a result of the method 200, wherein the result indicates that the test subject has clear vision (e.g., 20/20 vision) or vision that is clearer than can be determined using the method 200. The general vision result may not state a particular property of the test subject's vision aside from indicating a range or lower limit to the test subject's vision. For example, the test subject may have better vision than 20/20, and the general vision result indicates that the test subject has at least 20/20 vision without reporting how much better the test subject's vision is compared to 20/20 vision (e.g., exactly 20/15 or 20/10 vision). Thus, the method 200 can determine that a test subject's vision is sharper than the testing environment, display, or target image can precisely ascertain.

In cases where subjective recognition is finally not achieved, either after modification of the target image's color (in block 208) or via modification of the scale or rotation of the target image (via blocks 216 and 218), the last distance at which there is subjective recognition, before the visual element(s) are no longer recognized, is referred to as the maximum distance of best acuity (MDBA). Since at least part of the method 200 includes testing acuity by changing spectral colors instead of by changing distance, the MDBA can account for the diopter offset induced by the chromatic aberration of the eye of the test subject. Thus, the simulated distance between the test subject 308 and the electronic device (e.g., 306c, the effective distance at which the final recognized color-shifted target image elements are discerned) can be used as the MDBA when spectral color shifting has been used to find the final point of subjective recognition for the test subject.

In some examples, the method 200 can include determining at least one parameter of the eye of the test subject (e.g., spherical power or cylindrical power) based on the actual distance (e.g., 306b) and the second spectral color. The effective or virtual distance 306c to the test subject can be calculated from distance 306b and the degree of color change (e.g., wavelength change) from the first target image 400 to the second target image 500 (along with any further factors related to scaling or rotating the target image). A vision test result, such as a spherical power of the test subject's eyes, a visual acuity value (e.g., “20/30 vision” or “−1.5 diopter”), a cylinder power and/or axis, or similar eye parameters, can be produced, recorded, and/or reported.

In some examples, the method 200 can further include outputting the determined or calculated eye parameter of block 212 (or the general vision result 214) to the test subject (e.g., via a graphical user interface or speaker) or transmitting a signal indicating the eye parameter or result to a remote server (e.g., server 127), a healthcare professional, or database. As explained herein, the information can be used to form or manufacture a lens (e.g., eyeglasses or contact lenses) for the test subject that accounts for the test subject's eye properties determined during the refraction.

In various embodiments, the target images 400, 500 can be used to determine cylindrical parameters of an eye of a test subject. The target images can be presented at various angles of rotation, and the test subject can provide input regarding whether they recognize the properties of the visual elements therein. For example, the parallel line features 410/412 (and center field 414) or 510/512 (and center field 514) can be blurred to a single line at a certain angle of rotation for a single distance from the test subject. The spectral color of elements of the target image can be shifted to find MDBA for various rotated positions relative to the test subject's eye. The MDBA values for those angles can be aggregated to determine visual acuity along various axes for the eye of the test subject. Thus, various angles of rotation can provide information regarding cylindrical power of the eye and/or cylindrical axis of the eye by comparing physical distances and chromatic shift values for multiple different rotated angles of the target image(s).

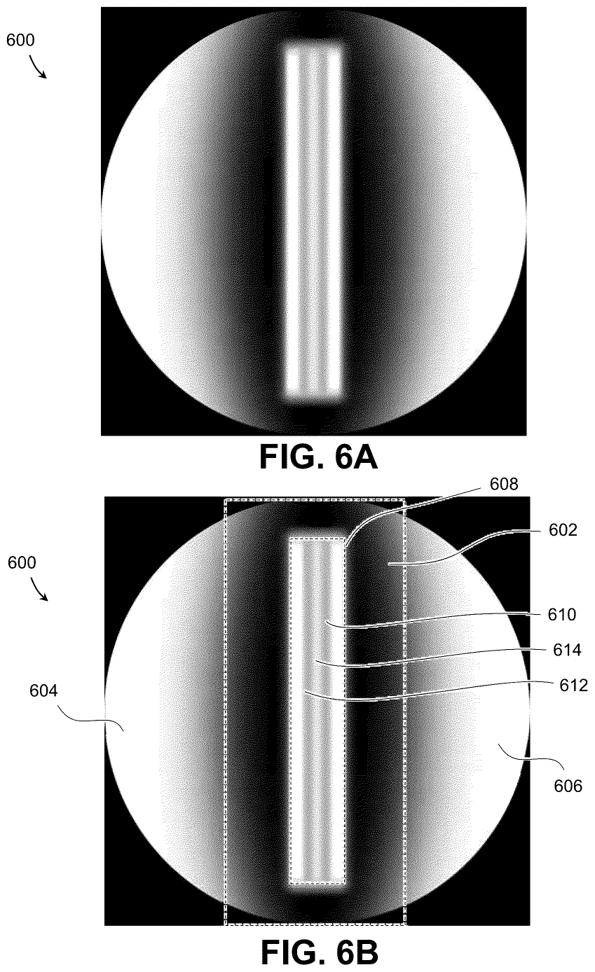

In some examples, the target images 400, 500 can be displayed with inverted contrast. For example, target image 600 of FIG. 6A shows the first target image 400 with inverted values, wherein the indicator numerals in FIG. 6B refer to corresponding features of FIG. 4B. The parallel line features 610, 612 are positioned adjacent to and within a relatively light or white background box 608 and with a relatively bright center field 614. The background box 608 is surrounded by a darker central portion 602 and lighter left and right portions 604, 606. The method 200 can therefore display target images having different contrast levels or relative contrast between visual elements as compared to the target images 400, 500 of FIGS. 4A-5B. Testing using reversed contrast or varied contrast can help identify eye parameters more effectively for certain test subjects and in certain testing environments.

Furthermore, in some examples, a method 700 may be implemented (e.g., by system 100) to determine eye parameters using, in part, changes in distance, contrast, and spectral color of a target image. For example, as shown in FIG. 7, the method 700 may include displaying a target image 702. The target image (e.g., 400, 600) can be shown at a distance from the test subject (e.g., at arm's length from the test subject's face, at 2 meters (6.1 feet) away from his or her face, or the maximum distance available to the test subject). The test subject may modify the distance to the target image (e.g., as shown in block 704) if needed to reach the needed testing distance.

The distance from the test subject can be used to modify the contrast in the target image 400, 600, as shown in block 706. For example, the test subject may be presented a target image having relative contrast values between in-image elements, e.g., between visual elements 402, 404, 406, 408, 410, 412, and 414 (or corresponding elements of FIG. 6) having a first set of values or ratios when the target image is presented at a first distance and to have a second set of values or ratios when the target image is presented at a second distance. In some examples, the contrast values or ratios can be modified corresponding to movement of the test subject relative to the target image (e.g., in connection with blocks 704 and 706). Additionally, contrast levels can be adjusted based on scaling of the target image, wherein target images that are displayed scaled up in size or dimensions are shown with lower contrast than target images displayed scaled down or not scaled at all but positioned at a greater distance from the test subject (e.g., similar to blocks 216 and 218). For example, a target image may be scaled to a larger size on a display screen when the display is farther from the test subject as compared to when the same display is nearer to the test subject, and contrast can follow the inverse, with lower contrast for larger scaled images and higher contrast for smaller scaled images. Contrast adjustments can change the difficulty of discernment of the target image elements for the test subject, with higher contrast generally reducing difficulty and lower contrast generally increasing difficulty for any given testing distance.

Additionally, as shown in block 708, colors of one or more elements of the target image can be modified upon finding a maximum distance at which visual elements at a certain contrast setting remain visible to the test subject. Due to contrast adjustment, as discussed in connection with block 706, the chromatic changes implemented in connection with block 708 can be more limited in the number of changes required as compared to embodiments where contrast is held constant at all distances from the test subject. Nevertheless, the color changes of the target image elements can affect the diopter offset of the image elements for the test subject, similar to embodiments discussed above.

Block 710 can include detecting subjective recognition. The test subject can provide input regarding his or her perception of target image elements (e.g., seeing two parallel line features or one line feature of the two lines blurred with a center field between the lines). If subjective recognition is not provided, the method 700 may continue to determine an eye parameter in block 712, similar to block 212, accounting for the effect on the MDBA by the distance to the target image, the contrast of the target image, and the color shift of the target image when determining the parameter of the eye (e.g., the spherical or cylindrical power of the eye).

If subjective recognition is detected/input by the test subject in block 710, the method can proceed by modifying target image contrast via block 706, modifying the target image color via block 708, or producing a general vision result (similar to block 214, as shown in block 714). Thus, the perception of the test subject can be re-tested under different contrast or chromatic conditions for the target image, via block 710, until reaching a state in which the test subject changes from subjective recognition to a lack of subjective recognition. At that state, the distance to the target image and/or its effective distance (as affected by contrast and chromatic shifts) can be used to determine at least one eye parameter in block 712. Otherwise, if the limits of the test are reached without a loss of subjective recognition, the general vision result can be produced in block 714. Furthermore, the determined eye parameter (or general vision result) can be transmitted to a server or to a healthcare professional, output to the test subject, or otherwise dispatched, as described above.

In some examples, the determined eye parameters can be applied to physical components such as by forming/manufacturing lenses for eyeglasses or contact lenses or by identifying appropriate lenses or contact lenses. Thus, the results of the methods 200, 700 can be used for refraction of a test subject and then implemented to provide vision-corrective lenses for the test subject.

In some examples, the method (e.g., 200) may include modifying a rotation of the second target image (e.g., 500) and determining whether the test subject recognizes the property of the second visual element (e.g., parallel line features 510, 512) of the second target image at the modified rotation. In some cases, modifying the rotation (e.g., via block 218) may comprise incrementing the rotation by about 15 to about 45 degrees per iteration. For instance, the rotation may be incremented by about 15 to about 20 degrees, or by about 35 to about 45 degrees, depending on the testing protocol.

In some embodiments, the electronic device (e.g., system 100 or 302) may include an ambient light sensor, and the method (e.g., 200, 700) may include detecting an ambient light brightness condition and adjusting a contrast or intensity of the first target image (e.g., 400) based on the detected ambient light brightness condition. For example, a bright ambient environment for the test subject (e.g., 308) can be detected, and the processor (e.g., 102) can cause the display (e.g., 132, 304) to present a target image with greater brightness or contrast as compared to when a relatively dark ambient environment is detected.

In various embodiments, the parameter of the eye determined by the method (e.g., 200, 700) may comprise at least one of a spherical power, a cylindrical power, or a cylindrical axis. The parameters can be used to characterize the refractive properties of the test subject's eye (e.g., properties of the compound lens including ocular lens 310) and may be used in prescribing corrective lenses.

In some examples, the method (e.g., 200, 700) may further comprise outputting the determined parameter of the eye (e.g., via block 212 or 712) to the test subject (e.g., 308) via a graphical user interface (e.g., displayed on display 132, 304) or transmitting a signal indicating the determined parameter of the eye to a remote server (e.g., server 127). The remote server may be associated with a healthcare professional or a database for storing vision test results. In some cases, the determined parameter may be directly transmitted to a healthcare professional.

In some embodiments, determining the parameter of the eye (e.g., via block 212) may comprise calculating an effective distance (e.g., 306c) based on the actual distance (e.g., 306b) and a diopter offset induced by chromatic aberration of the eye of the test subject (e.g., 308). The effective distance accounts for the shift in focal point (e.g., 313) caused by the spectral color change, allowing the system (e.g., 100, 302) to determine the test subject's visual acuity as if the target image were positioned at the effective distance rather than the actual physical distance.

In some examples, the second spectral color (e.g., of target image 500) may shift dioptric power by about −0.5 D to about −0.75 D relative to the first spectral color (e.g., of target image 400). This shift can be achieved by changing from white or grayscale light (with median densities in the greens of about 513 nm average wavelength) toward blue light at about 450 nm to about 480 nm average wavelength. The magnitude of the dioptric shift may depend on the degree of spectral color change applied to the target image.

In some embodiments, the method (e.g., 200, 700) may further comprise forming a lens for eyeglasses or contact lenses based on the determined parameter of the eye. The determined eye parameters can be applied to physical components by manufacturing lenses or by identifying appropriate pre-made lenses or contact lenses for the test subject (e.g., 308).

In some examples, the distance (e.g., 306b) at which the target image (e.g., 400, 500) is displayed may be a maximum distance available in a testing environment due to physical constraints of a testing location. For instance, the maximum distance may be limited by the size of a room, the presence of walls or other obstacles, or the length of the test subject's arm when holding the electronic device (e.g., 302). The color shifting techniques described herein (e.g., via block 208) can extend the effective testing range beyond this physical maximum distance.

In some embodiments, the method (e.g., 200) may include, prior to determining that the test subject does not recognize the property of the second visual element (e.g., 510, 512), determining that the test subject recognizes the property of the second visual element at the distance (e.g., 306b). In response, the method may include further modifying the second spectral color (e.g., via block 208) to produce a third target image having a third spectral color, and then determining that the test subject does not recognize a property of a third visual element of the third target image at the distance. This iterative process of color modification can continue until the test subject (e.g., 308) no longer subjectively recognizes the property of the visual element being tested.

In some examples, the sensor (e.g., 114) used to detect the distance between the display (e.g., 132, 304) and the test subject (e.g., 308) may comprise an infrared emitter (e.g., 139) and detector configured to determine the distance via time-of-flight information. The emitter may output infrared light, and the detector (e.g., sensor 114) may sense the reflected light to calculate the distance based on the time elapsed between emission and detection.

In some embodiments, a method (e.g., 700) may include displaying a target image (e.g., 400, 600) to a test subject (e.g., 308) via a display (e.g., 132, 304) at a distance from the test subject, modifying a contrast of the target image based on the distance (e.g., via block 706), determining that the test subject recognizes a property of the visual element (e.g., 410, 412) of the target image at the distance and the modified contrast (e.g., via block 710), modifying a spectral color of the target image (e.g., via block 708), determining that the test subject does not recognize the property of the visual element of the target image at the modified spectral color, and determining a parameter of an eye of the test subject based on the distance, the modified contrast, and the modified spectral color (e.g., via block 712). In some cases, modifying the contrast may comprise decreasing the contrast when the target image is scaled to a larger size on the display. For example, a target image shown with relatively large real-world dimensions may be presented with lower contrast, while a target image shown with relatively small real-world dimensions may be presented with higher contrast.

Various inventions have been described herein with reference to certain specific embodiments and examples. However, it will be recognized by those skilled in the art that many variations are possible without departing from the scope and spirit of the inventions disclosed herein, in that those inventions set forth in the claims below are intended to cover all variations and modifications of the inventions disclosed without departing from the spirit of the inventions. The terms “including” and “having” as used in the specification and claims shall have the same meaning as the term “comprising.”

As used herein, conjunctive terms (e.g., “and”) and disjunctive terms (e.g., “or”) should be read as being interchangeable (e.g., “and/or”) whenever possible. Furthermore, in claims reciting a selection from a list of elements following the phrase “at least one of,” usage of “and” (e.g., “at least one of A and B”) requires at least one of each of the listed elements (i.e., at least one of A and at least one of B), and usage of “or” (e.g., “at least one of A or B”) requires at least one of any individual listed element (i.e., at least one of A or at least one of B).

What is claimed is:

1. A computer-implemented method for interactively determining an ocular parameter of an eye of a test subject, the method comprising:
displaying a first target image to a test subject via a display, the first target image including a first visual element comprising a first spectral color;
determining that the test subject recognizes a property of the first visual element of the first target image at a distance from the display;
displaying a second target image to the test subject, the second target image including a second visual element comprising a second spectral color;
determining that the test subject does not recognize a property of the second visual element of the second target image at the distance from the display; and
determining a parameter of an eye of the test subject based on the distance and the second spectral color.

2. The method of claim 1, wherein the first visual element comprises a set of lines having a first brightness and a first background having a second brightness, the first brightness being different from the second brightness.

3. The method of claim 2, wherein the first brightness is about ten times brighter than the second brightness.

4. The method of claim 2, wherein the second visual element comprises a second set of lines having a third brightness and a second background having a fourth brightness, the third brightness being different from the fourth brightness.

5. The method of claim 4, wherein the second visual element comprises a border portion surrounding the second background, the border portion having a border brightness different from the fourth brightness.

6. The method of claim 1, wherein the first visual element comprises at least two separate elements set on a background and a border surrounding the background, the background having a first brightness, the border having a second brightness different from the first brightness.

7. The method of claim 6, wherein the border has a different spectral color relative to the at least two separate elements.

8. The method of claim 1, wherein the first spectral color of the first visual element is substantially gray.

9. The method of claim 1, wherein the second spectral color of the second visual element has a blue-shifted wavelength relative to the first spectral color of the first visual element.

10. The method of claim 1, wherein the second spectral color shifts dioptric power of the second target image relative to the first target image.

11. The method of claim 1, wherein the property of the first visual element is a separation between two parts of the first visual element, and wherein the property of the second visual element is a separation between two parts of the second visual element.

12. The method of claim 1, further comprising:
prior to displaying a second target image to the test subject, displaying an intermediate target image to the test subject, the intermediate target image including a third visual element comprising a third spectral color; and
determining that the test subject recognizes a property of the third visual element of the intermediate target image at the distance from the display.

13. The method of claim 1, further comprising:
determining the distance from the display;
scaling a size of the first target image according to the distance; and
scaling a contrast between portions of the first visual element according to the distance.

14. The method of claim 1, wherein the second spectral color comprises a blue: red ratio in a range of about 4:1 to about 20:1 in RGB values.

15. The method of claim 1, wherein the second spectral color comprises a blue: green ratio in a range of about 3:1 to about 5:1 in RGB values.

16. An apparatus for interactively measuring an ocular parameter, the apparatus comprising:
a display configured to display to a test subject a user interface, a first target image, and a second target image;
a sensor operable to detect a distance between the display and the test subject;
a processor;
a non-transitory, computer-readable memory device comprising instructions encoded thereon that, when executed by the processor, cause the processor to:
trigger the display to show a first target image to a test subject, the first target image including a first visual element comprising a first spectral color;
determine, via the sensor, a distance from the display to the test subject;
determine, via the user interface, that the test subject recognizes a property of the first visual element of the first target image at the distance;
trigger the display to show a second target image to the test subject at the distance, the second target image including a second visual element comprising a second spectral color;
determine, via the user interface, that the test subject does not recognize a property of the second visual element of the second target image at the distance from the display; and
determine a calculated parameter of an eye of the test subject based on the distance and the second spectral color.

17. The apparatus of claim 16, wherein the first visual element comprises a set of lines having a first brightness and a first background having a second brightness, the first brightness being different from the second brightness.

18. The apparatus of claim 16, wherein the processor is further configured to:
prior to displaying a second target image to the test subject, display an intermediate target image to the test subject, the intermediate target image including a third visual element comprising a third spectral color; and
determine that the test subject recognizes a property of the third visual element of the intermediate target image at the distance from the display.

19. A non-transitory computer-readable storage medium comprising computer-executable instructions operable to, when executed by at least one computer processor, enable the at least one computer processor to cause at least one computing device to:
display a first target image to a test subject via a display, the first target image including a first visual element comprising a first spectral color;
determine a distance from the display to the test subject;
determine that the test subject recognizes a property of the first visual element of the first target image at the distance;
display a second target image to the test subject at the distance, the second target image including a second visual element comprising a second spectral color;
determine that the test subject does not recognize a property of the second visual element of the second target image at the distance from the display; and
determine a calculated parameter of an eye of the test subject based on the distance and the second spectral color.

20. The non-transitory computer-readable storage medium of claim 19, wherein the first visual element comprises a set of lines having a first brightness and a first background having a second brightness, the first brightness being different from the second brightness.
